(19) United States
(12) Patent Application Publication
(10) Pub. No.: US 2012/0150000 A1
(43) Pub. Date: Jun. 14, 2012

(54) Non-Invasive Monitoring Device

(76) Inventors: Ahmed Al-Shamma'a, Liverpool (GB); Alex Mason, Liverpool (GB); Andrew Shaw, Liverpool (GB)

(21) Appl. No.: 13/320,002

(22) PCT Filed: May 11, 2010
(86) PCT No.: PCT/GB2010/050766

§ 371 Date: Feb. 27, 2012

(30) Foreign Application Priority Data
May 11, 2009 (GB) 0908043.3

(51) Int. Cl. A61B 5/145

(52) U.S. Cl. 600/309

(57) ABSTRACT

The invention concerns a device for non invasive monitoring of the concentration of a constituent of a human or animal bloodstream. In the preferred example the device comprises drive circuitry (50) for provision of an alternating current at a microwave frequency. This frequency is adjustable. The drive circuitry may for example comprise a voltage controlled oscillator. The device has a sensor (19) adapted to be placed in proximity to the body of the human or animal, the sensor being electrically connected to said drive circuitry to receive said alternating current and being adapted to project microwave energy into the said body. Detector circuitry is provided for detecting a signal transmitted and/or reflected by the sensor, the detected signal properties being dependent on the concentration of the said blood constituent.

The present invention relates to non-invasive monitoring of blood constituents. It is applicable in particular, but not exclusively, to the monitoring of the concentration of glucose in the bloodstream of a person or animal.

There are various practical situations in which it is necessary to determine, quantitatively or merely relatively, the concentration of a chosen substance, or of chosen substances, in the blood. A very important example is monitoring of blood glucose levels in those unfortunate enough to suffer from diabetes mellitus. The disease results from diminished production of the hormone insulin (in its Type 1 form) or from resistance to insulin's metabolic effects (the Type 2 form of the disease). Both can lead to hyperglycaemia—an excessive concentration of glucose in the blood—and so to various immediate symptoms including excessive urine production, lethargy and changes in energy metabolism. Acute complications include hypoglycaemia (excessively low levels of blood glucose), ketoacidosis and even coma. Long term implications of untreated diabetes include cardiovascular disease, chronic renal failure and retinal damage.

Treatment regimes normally include administration of insulin, which can be delivered for example using a syringe, an insulin pump or an insulin pen. Timing and dosage of insulin supplements are typically to be adjusted on the basis of measured blood glucose levels. The patient him or herself is trained to carry out the necessary measurement procedure at suitable time intervals, and to dose herself as necessary. Monitoring is frequent, so a straightforward, rapid and preferably painless means for determining blood glucose concentration is highly desirable, both commercially and from the point of view of the sufferer's health and well being.

One widely used method involves obtaining a small blood sample by piercing the skin, typically the finger, in order to draw a drop of blood onto a disposable chemical strip which reacts with the blood to produce a colour change indicative of the glucose level. Electronic non-disposable meters are also available which measure the electrical characteristics of a blood sample in order to provide a reading. Obviously “invasive” tests of these types, reliant on the production of a blood sample, are inconvenient and potentially even painful for patients.

Attempts at providing a “non-invasive” test, not reliant on the production of a blood sample, have included research based on:
(a) use of near infra red radiation—e.g. Omar Amir, Daphna Weinstein, Silviu Zilberman et al, “Continuous Noninvasive Glucose Monitoring Technology Based On Occlusion Spectroscopy”, journal of Diabetes Science Technology, Volume 1, Issue 4, July 2007
(b) use of ultrasound—e.g. Joseph Kost, Samir Mitragotri, Robert Gabbay, Michael Pishko, Robert Langer, “Transdermal Monitoring of Glucose and Other Analytes Using Ultrasound”, Nature Journal, Issue 6, pp 347-350, 2000
(c) dielectric spectroscopy—Buford Randall Jean, Eric C. Green, Melanie J. McClung, “A Microwave Frequency Sensor for Non-Invasive Blood-Glucose Measurement”, IEEE Sensors Applications Symposium, Atlanta, February 2008.

United Kingdom patent application (3B2428093 (Hancock and Microoncology Ltd) describes an instrument for non-invasive monitoring of blood glucose using low power emitted energy in the microwave region of the spectrum, using an antenna arrangement to provide the microwave emission.

While prior art systems do show a reaction to differing concentrations of glucose, it is often unclear whether such reactions are predictable or repeatable.

Although the above discussion is focused on monitoring of blood glucose levels, there are important situations in which other blood constituents need to be measured. Law enforcement agencies, for example, have a need to measure blood alcohol levels of drivers in order to establish whether legal limits have been breached. Sports authorities test for many different banned substances in athletes' bloodstreams, and the concentration of naturally occurring blood constituents other than glucose are monitored for medical purposes. Warfarin can be administered medicinally and the blood's warfarin concentration may then need to be monitored.

In accordance with the present invention, there is a device for non invasive monitoring of the concentration of a constituent of a human or animal bloodstream, the device comprising

drive circuitry for provision of an alternating current at a microwave frequency;

adjustment circuitry for adjustment of the said frequency of said alternating current;

a sensor adapted to be placed in proximity to the body of the human or animal, the sensor being electrically connected to said drive circuitry to receive said alternating current and being adapted to project microwave energy into the said body; and

detector circuitry for detecting a signal transmitted and/or reflected by the sensor, the detected signal properties being dependent on the concentration of the said blood constituent.

It must be understood that where circuitry is referred to, this may in principle be either of analogue or digital type. However in one embodiment the drive circuitry comprises an oscillator. Specifically the oscillator may be a voltage controlled oscillator. In this case the adjustment circuitry may comprise a source of an adjustable voltage for supply to the voltage controlled oscillator to control it.

The microwave frequency is preferably adjustable within a range from 1 to 6 GHz. More preferably the frequency is adjustable within a range from 1.5 to 3.5 GHz. More preferably still the frequency is adjustable within a range from 3.1 to 3.4 GHz. The adjustment of the frequency need not be continuous: in some embodiments a limited set of discrete frequencies only are used.

In a preferred embodiment the sensor comprises a ring resonator.

It is particularly preferred that the sensor comprises a conductive path interrupted by a discontinuity.

A sensor of this type can be designed to be sensitive to the dielectric properties of material (the body part) placed in the vicinity of the discontinuity, and to be relatively insensitive to the placement of material at other regions of the sensor. The conductive path may lead from a sensor input, connected to the drive circuitry to receive the alternating current, to a sensor output. Preferably the sensor has conductive elements forming two separate limbs leading from input to output, the discontinuity being formed in one of them.

The discontinuity may be formed in a conductive loop. In some embodiments this can loosely be referred to as a resonant loop, taking account of the frequency of the alternating current and the loop's dimensions.

Preferably the conductive path is juxtaposed with a ground element. Where the conductive path comprises a resonant loop, the ground element may comprise a first ground element surrounding the conductive loop and a second ground element within the loop. the first and second ground elements being electrically connected by a conductor passing through the aforesaid discontinuity in the loop.

It is particularly preferred that the sensor is a coplanar waveguide.

It is particularly preferred that the sensor comprises conductors arranged to forma capacitance connected to the drive circuitry and to the detector circuitry, so that the dielectric properties of a body part placed in the vicinity of the said capacitance are represented in the detected signal.

Preferably the aforesaid conductive paths of the sensor are formed on a dielectric substrate. The substrate may be rigid, and may take the form of a circuit board. The substrate may be flexible for conformity with and/or placement around the body part. For example it may take the form of a cuff for placement around a person's wrist.

The device preferably further comprises signal processing circuitry for receiving the output of the detector circuitry and for providing an indication of the concentration of the said blood constituent. While the signal processing circuitry may again in principle be of analogue or digital type, it will typically comprise a microprocessor. Preferably it comprises a trained neural net. It may be sensitive to any one or more of a frequency of a feature of the detected signal, phase of the detected signal, power of the detected signal and amplitude of the detected signal. All of these properties may be indicative of the concentration of the blood constituent. Preferably the signal processing circuitry is sensitive to all of these properties.

Specific embodiments of the present invention will now be described, by way of example only, with reference to the accompanying drawings, in which:

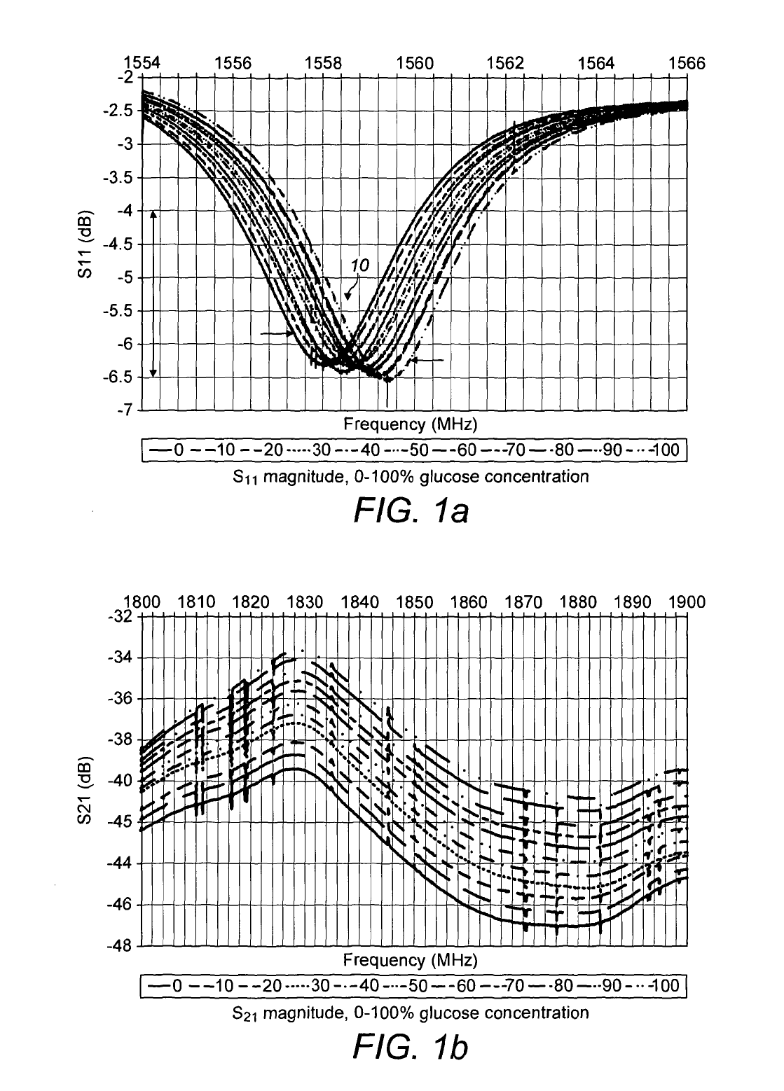

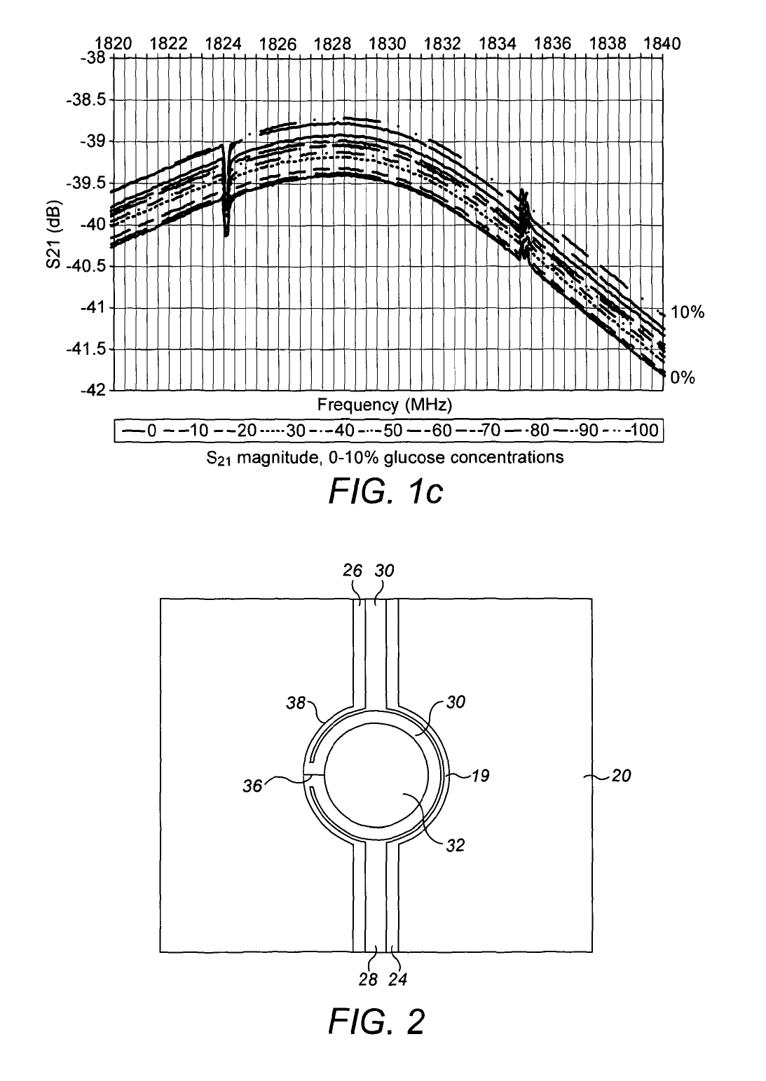

FIGS. 1a-c are graphs of measured signal amplitude (on the vertical axis) in a resonant cavity of an apparatus embodying aspects of the present invention over a range of frequencies (on the horizontal axis);

FIG. 2 is a plan view of a sensor for use in the present invention;

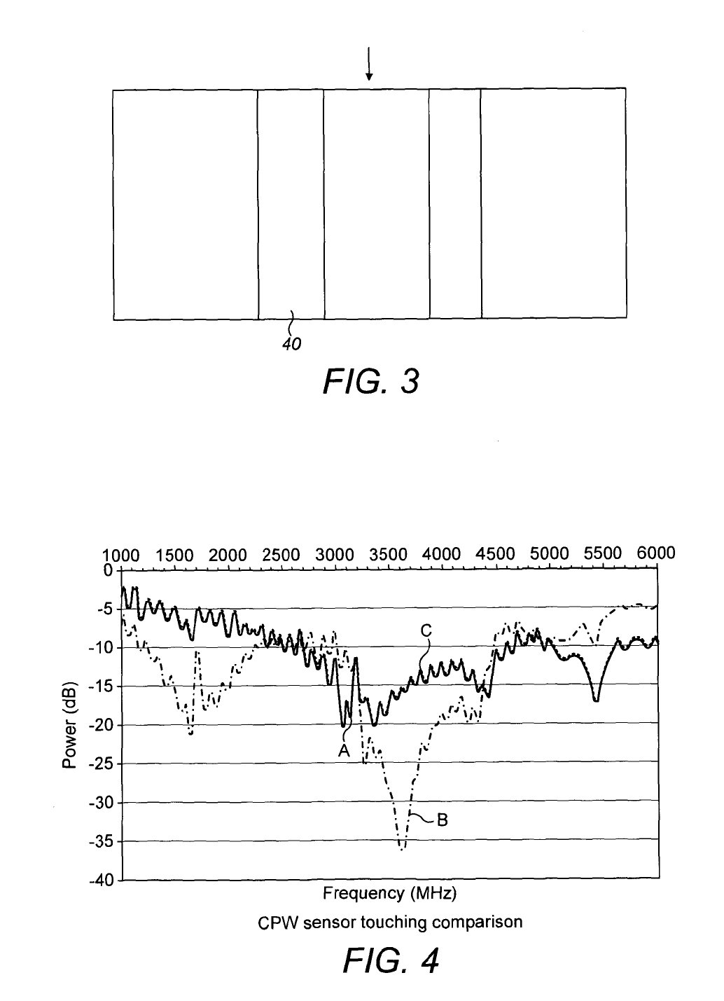

FIG. 3 is an enlarged view of a portion of the sensor, on which electrical charges and lines of electrical field are indicated;

FIG. 4 is a graph of transmitted signal power against frequency obtained using the sensor and showing how the power changes when the sensor is touched;

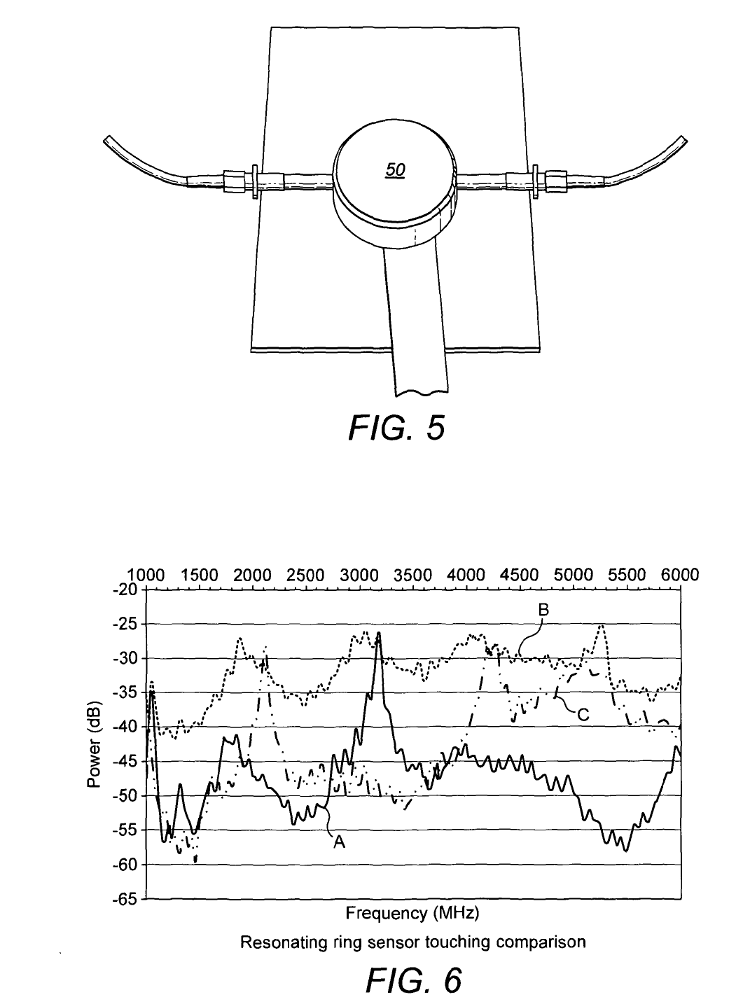

FIG. 5 illustrates a resonating ring structure;

FIG. 6 is a graph similar to FIG. 4 but obtained using the resonating ring structure of FIG. 5;

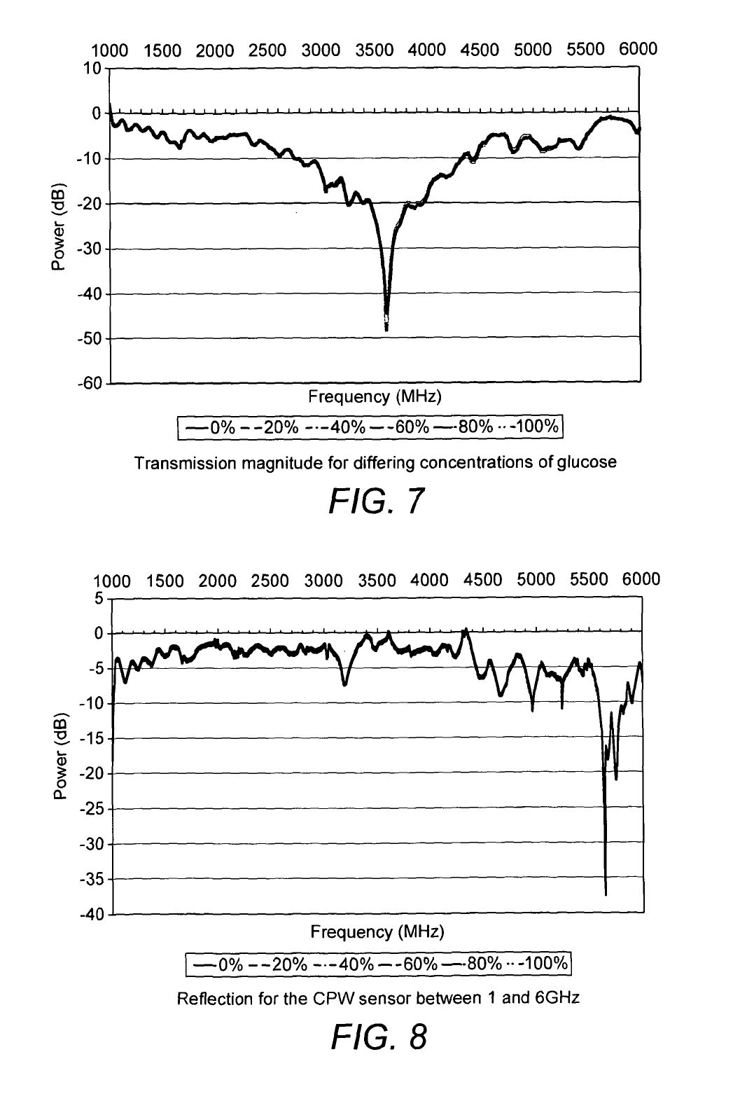

FIG. 7 is a graph of power transmitted from the FIG. 2 sensor over a broad range of frequencies;

FIG. 8 corresponds to FIG. 7 except that it shows power reflected by the sensor;

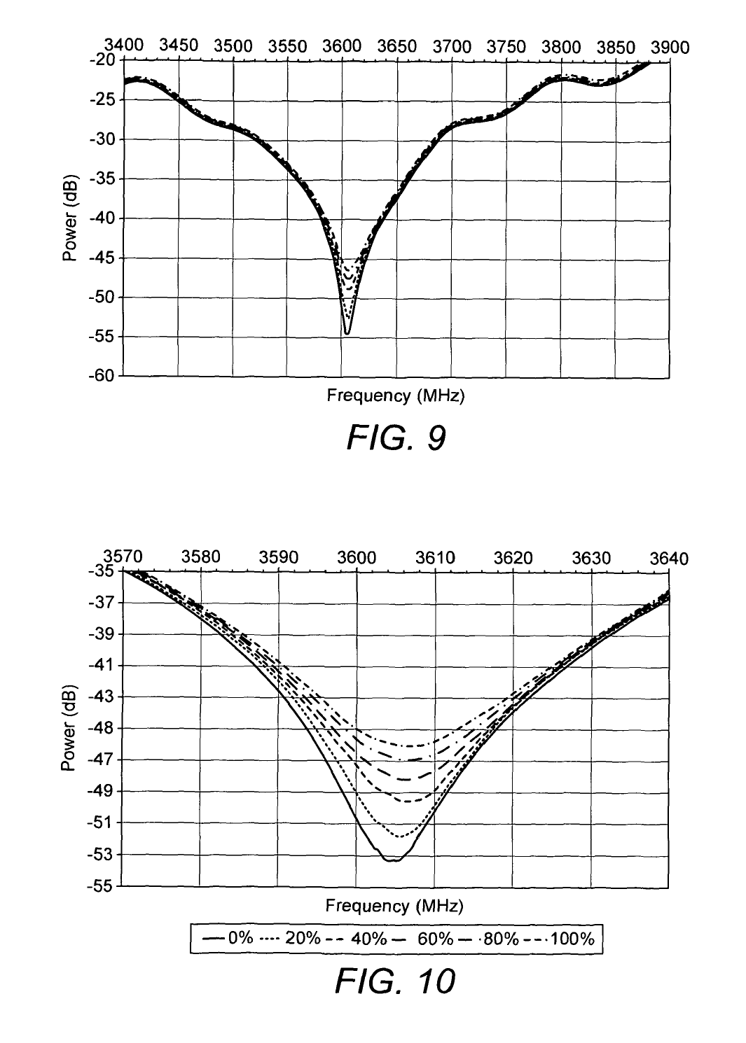

FIG. 9 is a graph of power transmitted from the sensor over a selected frequency range;

FIG. 10 is a graph of power transmitted from the sensor over a still narrower frequency range;

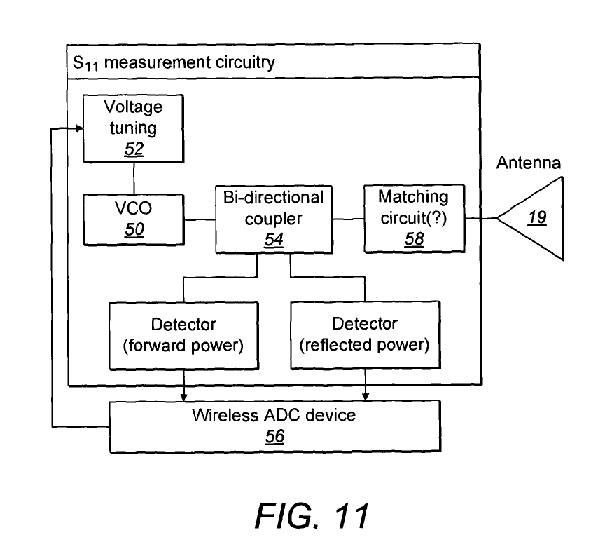

FIG. 11 is a schematic representation of an electronic circuit for driving the sensor and measuring transmitted and reflected power;

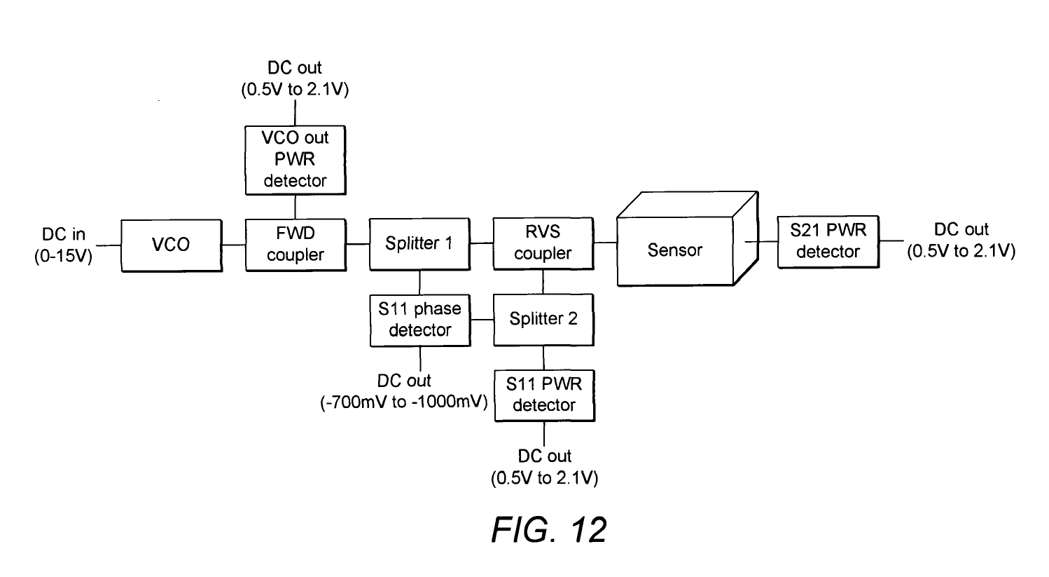

FIG. 12 is a block diagram of circuitry incorporating a sensor embodying the present invention; and

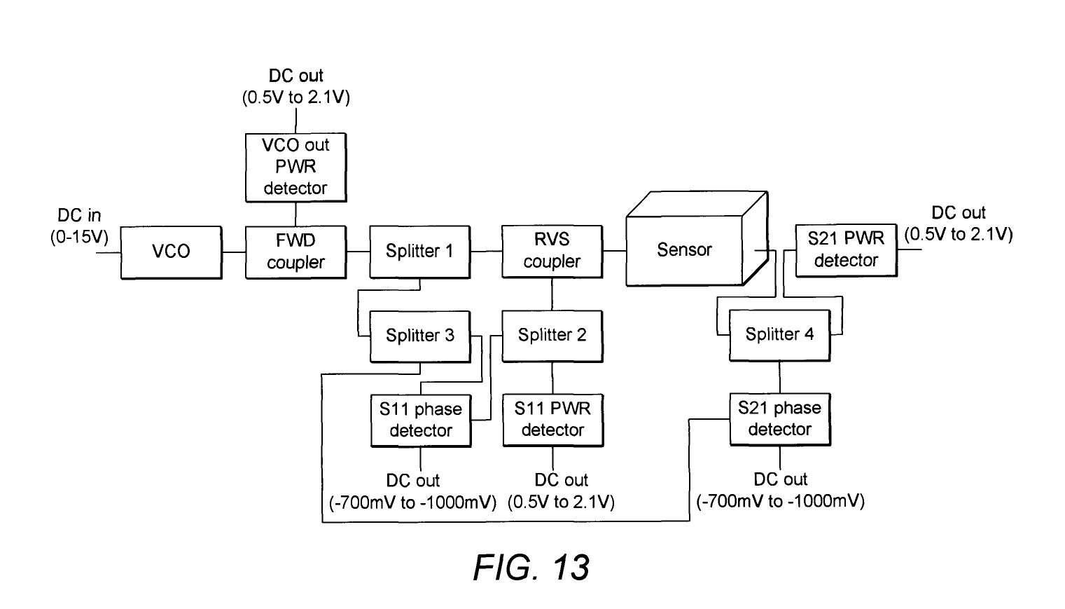

FIG. 13 is a modified version of the FIG. 12 diagram.

Initial testing of devices embodying the present invention was carried out using a microwave cavity (an enclosure defined by an electrically conductive wall, with dimensions chosen by reference to the intended microwave driving frequency) having an RF electrical input and a separate RF electrical output. These were attached to a commercially available Vector Network Analyser, used both (a) to provide the AC input signal to the resonator input, at a frequency which could be scanned over a chosen range, and (b) to measure, display and record over the chosen range of frequencies the magnitude of the power received at the output. Measurements were made of two different wave modes (S₁₁ and S₂₁). For each such trial a sample glucose solution of known concentration was placed in the microwave cavity, and multiple trials were carried out using glucose concentrations from zero to 1 Molar. Results are illustrated in FIGS. 1a (S₁₁ mode of the cavity) and 1b (S₂₁), each line in the graphs representing atrial with a different glucose concentration. FIG. 1c corresponds to FIG. 1b except that it shows a smaller frequency range, from 1820 to 1840 MHz, and a smaller range of glucose concentrations, from 0 to 10 percent. A pattern is observed that the output signal magnitude varies with glucose concentration. Also the frequency of the pronounced trough 10 in FIG. 1a, present even with a pure water sample, is seen to be modified by the presence of glucose, its shill being related to glucose concentration.

A microwave cavity is not necessarily well suited to use in a commercial blood glucose monitoring device, which should preferably be portable, easily applied to the skin and of convenient size. FIG. 2 illustrates a microwave radiating structure 19 intended for the purpose, based on the principle of co-planar waveguide (CPVV) feed design. The structure forms a sensor. It comprises shaped conductive tracks formed upon a dielectric substrate 20. In this particular example the substrate 20 is a circuit board of epoxy glass with relative permittivity of 4.4. In other embodiments different materials may be used to form the dielectric substrate, and in particular it may be flexible, to facilitate its placement against or around a chosen body part. Conductive metal layer 22 is cut away, in this example by etching in conventional manner, in regions 24 to form input and output ports 26, 28 connectable to drive and detection electronics. The ports 26, 28 are connected through a conductive loop 30, which in this particular example is circular. Within the conductive loop 30 and separated by a short radial distance from it is an inner ground plane 32, itself circular in this embodiment. Around the conductive loop 30 is an outer ground plane 34, likewise close to but radially separated from the conductive loop 30. A discontinuity 36 in the conductive loop 30 leaves room for a connection 38 between the inner and outer ground planes 32, 34. The underside of the dielectric substrate 20 also carries a ground plane, formed e.g. as a continuous metal layer, ensuring that power is radiated only from the upper side of the board, which in use is placed against the body of the individual being monitored.

The discontinuity provides a point in the circuit where power cannot simply be conducted from the input port 26 to the output port 28, so that some radiation must take place. As represented in FIG. 3, positive charge on the conductive loop 30 causes the ground plane 34 to become negatively charged. Electric field lines in the region between the two are seen at 40. Maxima and minima also form in these fields due to the application of high frequency alternating current. The example illustrated has dimensions of 52.5 mm width, 65 mm depth and 1.6 mm height (thickness).

In order to test the sensor 19, input port 26 was attached to the output of a Vector Network Analyser and output port 28 to the RF input of the same device. An initial trial involved the experimenter touching the sensor 19 with a fingertip in order to observe the effect on the power spectrum at its output port 28. Results are seen in FIG. 4, which contains:
Line A, obtained without touching the sensor,
Line B, obtained while touching the feed line (input) 26, and
Line C, obtained while touching the area of the discontinuity 36.

Significantly, lines A and B very largely coincide (in fact the former is largely concealed by the latter) indicating that contact of the experimenter's body with the feed had only a very slight effect on the measured spectrum, while contact in the area of the discontinuity 36 produced a dramatic and reproducible change. This is in contrast to results obtained, for the sake of comparison, using a simple ring resonator 50 as shown in FIG. 5. As seen in FIG. 6, the spectra A and B obtained with and without touching the feed line 51 of this structure are dramatically different. Touching the ring 52 in different places is likewise found to produce dramatic changes in the measured spectrum, making reproducible and meaningful results difficult to obtain.

As well as offering reproducible readings the sensor 19 has the advantage of emitting very little spurious radiation, which is desirable in microwave applications where circuits have to be in close proximity without interfering with one another.

In further trials of the sensor 19 a hollow plastic pipe (not shown) was placed across the discontinuity 36 of the sensor 19 and supplied with a flow of aqueous glucose solution. Results of various trials conducted using the arrangement are presented herein. FIG. 7 represents the power transmitted from input port 26 to output port 28 across a broad frequency range from 1 GHz to 6 GHz. FIG. 8 is similar but indicates the power reflected by the sensor 19 back to the source. Note that in FIG. 7 a pronounced change in signal amplitude with sample glucose concentration is observed at approximately 3.6 GHz, while FIG. 8 shows its own similar change at about 4.7 GHz. The region of interest around 3.6 GHz was investigated by sweeping through a narrower range of frequencies from 3400 MHz to 3900 MHz. The results for transmitted power are seen in FIG. 9 and these more clearly show that there is indeed a change in amplitude. From zero to 1 Molar glucose concentration, the change is of the order of 7 to 9 dB. FIG. 10 shows a repeat of these results using an even narrower frequency sweep (3570 to 3900 MHz) and it can be seen that as well as the amplitude change with glucose concentration there is also a small change in the frequency at which the minimum in the spectrum occurs. For glucose concentrations from zero to 0.4 Molar a 1 MHz shift in the minimum was observed.

The sensor 19 may, as already noted, use a flexible substrate in place of the epoxy glass circuit board 20, and may for example be formed as a cuff for placement around the wrist of a user. The wrist is chosen as a region benefiting from considerable blood flow, but other versions may be adapted for use at other locations on the body, such as a fingertip.

The Vector Network Analyser used in the above described laboratory trials is of course not suited to use in a commercial device. FIG. 11 is a schematic representation of a suitable circuit. A voltage controlled oscillator (VCO) 50 is used to provide the required microwave frequency AC signal, and its frequency is able to be swept over a limited range (chosen to taken in features of the spectrum indicative of glucose concentration such as the trough observed in FIGS. 9 and 10) by control of a tuning voltage supplied by circuitry 52. A bi-directional coupler 54 provides the facility to measure both the forward power from the VCO 50 and also the power reflected from the sensor 19. Both are fed to respective channels of an analogue to digital converter (ADC) 56, which in this example is a wireless device to transmit the digital data to a separate unit for storage and analysis. The ADC 50 is also used to control the tuning to provide e.g. a frequency sweep. Additional components 58 may be required for impedance matching between the coupler 54 and the sensor 19, although careful circuit design may allow these to be dispensed with. In some embodiments provision will additionally be made for sensing temperature at the measurement site, since glucose dielectric constant—and hence the measurements obtained—are known to be temperature dependent. Temperature measurements may be made with an infra red thermometer, or with other temperature sensing means.

Measurements which can be obtained by use of the above described sensors and circuitry include not only magnitude of the transmitted and reflected signals, over a range of frequencies, but also changes in signal phase which reflect dielectric properties of the material in the vicinity of the sensor, and specifically o f the blood flowing in the body part presented to the sensor. In order to be able to detect the relative phases of the input signal driving the sensor and (a) the transmitted signal or (b) the reflected signal, the arrangement seen in FIG. 12 is used. The oscillating signal for the sensor is provided by a voltage controlled oscillator 100. In the illustrated example, this is able to sweep through a range of frequencies from 3.2 GHz to 3.7 GHz when a 0-15 VDC sweep is applied to its input. The combination of a forward coupler 102 and a power detector 104 receiving the output of the voltage controlled oscillator make it possible to monitor performance of the voltage controlled oscillator. The power detector 104 gives a DC voltage output that reflects the measured power of the voltage controlled oscillator in dBm. A first splitter 106 splits the forward power coming from the voltage controlled oscillator 100 into two signals: one for the “S11” phase detector 108 and one that will supply the sensor with power. Between the first splitter 106 and the sensor, labelled 110 in this diagram, a reverse coupler 112 is inserted to provide the reflected signal from the sensor to a second splitter 114. The second splitter 114 divides its signal in two: one part is led to the S11 phase detector 108 while the other is led to an S112 power detector 116. The S11 power detector 116 measures the power reflected (in the S11 mode) from the sensor and sample. At the output of the sensor 110, an S21 power detector 118 measures power transmitted in the S21 mode—i.e. the power output of the sensor. If it is necessary additionally to detect the phase of the sensor's output, the arrangement seen in FIG. 13 can be used. A third splitter 120 has here been inserted between the first splitter 106 and the S11 phase detector 108 to provide an S21 phase detector 122 with the signal coming out of the voltage controlled oscillator 100. A fourth splitter 124 is interposed between the sensor 110 and the S21 power detector 118 and feeds the S21 phase detector 122 with the signal that has gone through the sensor. S21 phase is then the difference of phase between the signal going into the sensor and the signal coining out of the sensor.

The digitised data obtained is electronically stored for processing and retrieval. A software-implemented neural network, trained on suitable experimental data, may be used to interpret the data and to provide the required blood glucose concentration measurement. The unit may be in two parts, with a sensor transmitting data to a separate analysis/display module using the aforementioned wireless device, or the sensor, processing logic and display may be formed as a single unit.

The software for data analysis and prediction is split into two separate parts. A data analysis part pre-processes data obtained from the microwave sensor in order that data mining software can build a set of rules. Based upon these rules, the prediction software can then capture data from the sensor and determine the concentration of glucose. The data analysis software derives a number of values based upon the data it is given. These values are:
i. the mean and standard deviation of the data values;
ii. the frequency, f₀, at which S21 magnitude is at a minimum;
iii. the frequencies, f₁ and f₂ at which S21 magnitude is +3 dB (or double) that found at f₀. F₁ is smaller than f₀ and f₀ is smaller than f₂;
iv. the Q factor, which is defined as f₀ divided by f₂ minus f₁; and
v. the area above the curve between f₁ and f₂, which can be calculated from the data numerically.

The derived data is passed to the prediction software in which it is then possible to induce a rule free which can be used to determine concentration of the relevant blood constituent.

1. A device for non invasive monitoring of the concentration of a constituent of a human or animal bloodstream, the device comprising:
drive circuitry for provision of an alternating current at a microwave frequency;
adjustment circuitry for adjustment of the said frequency of said alternating current;
a sensor adapted to be placed in proximity to the body of the human or animal, the sensor being electrically connected to said drive circuitry to receive said alternating current and being adapted to project microwave energy into the said body; and
detector circuitry for detecting a signal transmitted and/or reflected by the sensor, the detected signal being dependent on the concentration of the said blood constituent.

2. A device as claimed in claim 1 in which the drive circuitry comprises a voltage controlled oscillator.

3. A device as claimed in claim 2 in which the adjustment circuitry comprises a source of an adjustable voltage for supply to the voltage controlled oscillator to control it.

4. A device as claimed in claim 1 in which the microwave frequency is adjustable within a range from 1 to 6 GHz.

5. A device as claimed in claim 1 in which the frequency is adjustable within a range from 1.5 to 3.5 GHz.

6. A device as claimed in claim 1 in which the frequency is adjustable within a range from 3.1 to 3.4 GHz.

7. A device as claimed in claim 1 in which the sensor comprises a ring resonator.

8. A device as claimed in claim 1 in which the sensor comprises a conductive path interrupted by a discontinuity.

9. A device as claimed in claim 8 in which the conductive path leads from a sensor input, connected to the drive circuitry to receive the alternating current, to a sensor output.

10. A device as claimed in claim 9 in which the sensor has conductive elements forming two separate limbs leading from input to output, the discontinuity being formed in one of them.

11. A device as claimed in claim 8 in which the discontinuity is formed in a conductive loop.

12. A device as claimed in claim 8 in which the conductive path is juxtaposed with a ground element.

13. A device as claimed in claim 11 comprising a first ground element surrounding the conductive loop and a second ground element within the loop, the first and second ground elements being electrically connected by a conductor passing through the aforesaid discontinuity in the loop.

14. A device as claimed in claim 1 in which the sensor is a coplanar waveguide.

15. A device as claimed in claim 8 in which the aforesaid conductive paths of the sensor are formed on a dielectric substrate.

16. A device as claimed in claim 15 in which the substrate is flexible for conformity with and/or placement around the body part.

17. A device as claimed in claim 16 in which the substrate takes the form of a cuff for placement around a person's wrist.

18. A device as claimed in claim 1 in which the sensor comprises conductors arranged to form a capacitance connected to the drive circuitry and to the detector circuitry, so that the dielectric properties of a body part placed in the vicinity of the said capacitance affect the detected signal.

19. A device as claimed in claim 1 which further comprises signal processing circuitry for receiving the output of the detector circuitry and for providing an indication of the concentration of the said blood constituent.

20. A device as claimed in claim 19 in which the signal processing circuitry comprises a trained neural net.

21. A device as claimed in claim 19 in which the signal processing circuitry is sensitive to any one or more of (a) a frequency of a feature of the detected signal, (b) phase of the detected signal, (c) power of the detected signal and (d) amplitude of the detected signal.

22. (canceled)
